(19) United States
(12) Patent Application Publication
(10) Pub. No.: US 2004/0197397 A1
(43) Pub. Date: Oct. 7, 2004

(54) Drug delivery system for treatment of urinary incontinence

(75) Inventor: Charles Ebert, Salt Lake City, UT (US)

Correspondence Address: THORPE NORTH & WESTERN, P.O. BOX 1219, SANDY, UT, 84091-1219, US

(73) Assignee: Watson Pharmaceuticals, Inc.

(21) Appl. No.: 10/654,262

(22) Filed: Sep. 2, 2003

Related U.S. Application Data
(60) Provisional application No. 60/407,009, filed on Aug. 30, 2002.

(51) Int. Cl. A61K 9/20

(52) U.S. Cl. 424/464

(57) ABSTRACT

Methods for the prevention or amelioration of urinary incontinence are disclosed and described. One method includes the coadministration of an anticholinergic agent with either an SSRI, or an SNRI, or both.

### PRIORITY DATA

This application claims priority to U.S. Provisional Patent Application Serial No. 60/407,009, filed on Aug. 30, 2002, which is incorporated herein by reference.

### FIELD OF THE INVENTION

The present invention relates to coadministration of an anticholinergic agent with either a selective serotonin reuptake inhibitor (SSRI) or a selective norepinephrine reuptake inhibitor (SNRI), or both, for the treatment of urinary incontinence. Accordingly, this invention covers the fields of pharmaceutical sciences, medicine and other health sciences.

### BACKGROUND OF THE INVENTION

Recently, an effective transdermal medication has been developed for the treatment of what has come to be known as overactive bladder which is occasioned by incontinence. Urge incontinence results from instability of the detrusor muscle, the muscle surrounding the bladder. The cholinergic receptors of the detrusor can be over-stimulated causing spasmodic contractions and a sensation of urgency to urinate, which may lead to an urgency to urinate, an increased micturation rate, and in extreme cases to incontinent episodes.

An oral sustained release technology is exemplified by Guittard et al., U.S. Pat. No. 6,262,115 (Alza) discloses tablets of oxybutynin without any further pharmaceutical component which has hydroxypropylmethylcellulose present in a molecular weight of approximately 10,000 (herein: Guittard). An effective transdermal delivery system has been developed by Watson Pharmaceuticals, Inc., which comprises technology disclosed in Quan et al., U.S. Pat. No. 5,834,010 (1998) (herein: “Quan”). Quan discloses transdermal technology for the delivery of oxybutynin. This application incorporates by reference in toto the complete disclosure of Quan. Quan teaches a transdermal medication that can be applied typically for twenty-four hours. It is recommended that such a transdermal medication be applied after a morning shower or bath, to thereby provide a twenty-four hour period of protection against such overactive bladder condition until the following morning. Other attempts to provide a treatment in this area include Pharmacia PCT application 0162236 with U.S. priority of Feb. 24, 2000, and Waki et al., European Patent Application 1174132 (2002). Waki et al. provide a recent time slice of the state of the art: “[T]he countermeasure for the bladder functional disorder such as urinary incontinence or pollakiuria associated with the increase in the population of the advanced age group is one of the most important question of vital interest in the medical treatment. Therefore, the development of the effective drugs in treating urinary incontinence or pollakiuria are to be desired, and various medicines in addition to oral drugs already available in the market are on their way to development. Oxybutynin hydrochloride used in the treatment of urinary incontinence and pollakiuria is well recognized as the excellent anticholinergic drug demonstrating its pharmacological effect through acetylcholine antagonism. An oral dosage form of the drug requires a comparatively small quantity of 2-3 mg per dose, but they have to be taken three times a day. In addition, the absorption of the drug through the intestinal tract is known to be good, but the higher hepatic metabolism is also reported (Pharmacopoeia 4 (5), 45-53, 1990). Regarding the routes of administration, the oral form has the advantage in not giving pain to patients as compared with the injection form, but it may not be easy to administer the medicine which has to be taken at the regular interval for the aged patients who may sometimes require the medical helper. Furthermore, the drug taken orally is inevitably absorbed into a hepatoportal vein through the intestinal tract, thereby being subjected to the first pass effect termed for the intense hepatic metabolism of the drug on its first passage and often leads to the marked decrease in biological availability in many cases. In order to maintain the effective concentration of the drug in the blood, it is necessary to administer a relatively large dose of drug, and as a result, an incidence in adverse effects naturally increases. From these standpoints, there is the urgent need for the development, of a preparation that is relatively easy to administer, long lasting in its effect, and yet with fewer adverse effects. In view of pharmacokinetics, a preparation that does not exhibit the behavior of a transitory drug concentration in the blood such that the blood concentration rapidly increases and then decreases as often observed in the general orally administrated preparation, but whose concentration increases gradually and its effective concentration in the blood can be continuously maintained over a long period of time is highly desired.”

In an embodiment of the invention that utilizes the Quan technology, in such an embodiment, the matrix patch comprises about 0.1% to about 50% by weight triacetin, more preferably about 1% to about 40% by weight triacetin, and most preferably about 2% to about 20% by weight triacetin. The polymer layer is preferably an adhesive, but can also be laminated to an adhesive layer or used with an overlay adhesive. Suitable polymers include acrylics, vinyl acetates, natural and synthetic rubbers, ethylenevinylacetate copolymers, polysiloxanes, polyacrylates, polyurethanes, plasticized weight polyether block amide copolymers, plasticized styrene-rubber block copolymers, and mixtures thereof. Acrylic copolymer adhesives are preferred. The matrix patch can also contain diluents, excipients, emollients, plasticizers, skin irritation reducing agents, carriers, and mixtures thereof provided that such additives do not alter the basic characteristics of the matrix patch.

In aspects of the invention utilizing the Quan technology, suitable polymers that can be used in the biocompatible polymeric layer of the matrix patch include pressure-sensitive adhesives suitable for long-term contact with the skin. Such adhesives must be physically and chemically compatible with the drug and enhancer, and with any carriers and/or vehicles or other additives incorporated into the drug/enhancer composition. Suitable adhesives for use in the matrix patch include acrylic adhesives including cross-linked and uncross-linked acrylic copolymers; vinyl acetate adhesives; natural and synthetic rubbers including polyisobutylenes, neoprenes, polybutadienes, and polyisoprenes; ethylenevinylacetate copolymers; polysiloxanes; polyacrylates; polyurethanes; plasticized weight polyether block amide copolymers, and plasticized styrene-rubber block copolymers. Preferred contact adhesives for use in the matrix patch herein are acrylic adhesives, such as TSR (Sekisui Chemical Co., Osaka, Japan) and DuroTak. RTM. adhesives (National Starch & Chemical Co., Bridgewater, N.J.), and polyisobutylene adhesives such as ARcare.TM. MA-24 (Adhesives Research, Glen Rock, Pa.).

In use, the matrix patch contains a distal backing laminated on the polymer layer. The distal backing defines the side of the matrix patch that faces the environment, i.e., distal to the skin or mucosa. The backing layer functions to protect the matrix polymer layer and drug/enhancer composition and to provide an impenetrable layer that prevents loss of drug to the environment. Thus, the material chosen for the backing should be compatible with the polymer layer, drug, and enhancer, and should be minimally permeable to any components of the matrix patch. Advantageously, the backing can be opaque to protect components of the matrix patch from degradation from exposure to ultraviolet light. Further, the backing should be capable of binding to and supporting the polymer layer, yet should be pliable to accommodate the movements of a person using the matrix patch. Suitable materials for the backing include metal foils, metalized polyfoils, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene and styrene-isoprene copolymers, polyethylene, and polypropylene. A thickness of about 0.0005 to 0.01 inch is preferred. The release liner can be made of the same materials as the backing, or other suitable films coated with an appropriate release surface.

The matrix patch can further comprise various additives in addition to the polymer layer, basic drug, and triacetin-containing penetration enhancer that are the fundamental components of the transdermal drug delivery system. These additives are generally those pharmaceutically acceptable ingredients that are known in the art of drug delivery and, more particularly, in the art of transdermal drug delivery provided that such additive ingredients do not materially alter the basic and novel characteristics of the matrix patch. For example, suitable diluents can include mineral oil, low molecular weight polymers, plasticizers, and the like. Many transdermal drug delivery formulations have a tendency to cause skin irritation after prolonged exposure to the skin, thus addition of a skin irritation reducing agent aids in achieving a composition that is better tolerated by the skin. A preferred skin irritation reducing agent is glycerin, U.S. Pat. No. 4,855,294.

The matrix patch device containing a polymer layer, the drugs, and triacetin-containing penetration enhancer is brought in contact with the skin or mucosa at a selected application situs and is held in place by a suitable pressure-sensitive adhesive. Preferably, the polymer layer of the matrix patch is an adhesive, but the polymer layer can also be laminated to an adhesive layer or used with an overlay adhesive.

While Quan provides an excellent medication for cases of overactive bladder for most patients, an improvement is contemplated in the present invention for post-menopausal women.

### SUMMARY OF THE INVENTION

Accordingly, the present invention provides an improvement in the method of providing a post-menopausal female patient with a weakened musculature in the area of the urinary tract with a greater resistance to active bladder reaction via the coadministration of a therapeutically effective amount of an anticholinergic agent with a therapeutically effective amount of an SSRI, or SNRI, or both. In one aspect, the anticholinergic agent may be oxybutynin and the SSRI may be fluoxitine.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### DETAILED DESCRIPTION

Accordingly, there are several specific aspects of the present invention. In a first embodiment, there is provided an improvement in the method of providing a post-menopausal female patient with a weakened musculature in the area of the urinary tract with an improved resistance to active bladder reaction via the oral delivery of oxybutynin, the improvement which comprises the coadministration therewith of an effective amount of FLUOXETINE whereby there is an enhanced resistance to said active bladder reaction.

In an aspect of this first embodiment, said coadministration is provided orally. In a still further aspect, the oral administration is via a sustained release vehicle to provide a 24 hour period of relief from active bladder reaction. In a further aspect, said coadministration is from a transdermal patch.

In a second aspect of the invention, an improvement is provided in the method of providing a post-menopausal female patient with a weakened musculature in the area of the urinary tract with an improved resistance to active bladder reaction via the oral delivery of oxybutynin, the improvement which comprises the coadministration therewith of an effective amount of paroxetine whereby there is an enhanced resistance to said active bladder reaction. In an embodiment, said coadministration is provided orally, and in a further embodiment thereunder, the oral administration is via a sustained release vehicle to provide a 24 hour period of relief from active bladder reaction. In an alternative embodiment of this aspect of the invention said coadministration is from a transdermal patch.

In a third aspect of the invention, an improvement is provided in the method of providing a post-menopausal female patient with a weakened musculature in the area of the urinary tract with an improved resistance to active bladder reaction via the oral delivery of Tolterodine, the improvement which comprises the coadministration therewith of an effective amount of fluoxetine whereby there is an enhanced resistance to said active bladder reaction. In an aspect of this embodiment, said coadministration is provided orally. In a still further aspect, the oral administration is via a sustained release vehicle to provide a 24 hour period of relief from active bladder reaction. In a further aspect, said coadministration is from a transdermal patch.

In a fourth aspect of the invention there is provided an improvement in the method of providing a post-menopausal female patient with a weakened musculature in the area of the urinary tract with an improved resistance to active bladder reaction via the oral delivery of Tolterodine, the improvement which comprises the coadministration therewith of an effective amount of paroxetine whereby there is an enhanced resistance to said active bladder reaction. In an aspect of this embodiment, said coadministration is provided orally. In a still further aspect, the oral administration is via a sustained release vehicle to provide a 24 hour period of relief from active bladder reaction. In a further aspect, said coadministration is from a transdermal patch.

In further aspects of the invention, oral and transdermal delivery systems are provided for each of the aspects of the invention set forth above.

The range of drugs in the composition of the invention will vary within amounts necessary to provide the desired effect of a prophylaxis or treatment of urinary incontinence in post-menopausal women with weakened musculature in the area of the urinary tract.

In oral formulation embodiments of the invention with oxybutynin it is contemplated that oxybutynin will be used in the form of its hydrochloride.

In sustained release formulations with any of oxybutynin, Tolterodine, Fluoxetine and Paroxetine, it is contemplated that twice the dosage will be provided vis a vis a regular (non-sustained release) tablet.

For Fluoxetine, the amount should vary from about 5 to about 120 mg. per dosage; in an embodiment, the range is 10 to 80 mg., and in an example the amount is 40 mg. A blood level that is continuously achieved for most of the period of delivery is to be achieved in accordance with the invention which should be from about 15 to 55 nanograms/ml;

For Paroxetine, the amount should vary from about 5 to 60 mg. per dosage unit; in an embodiment, the amount varies from about 10 to about 40 mg., and in a preferred embodiment the amount is 30 mg.

For oxybutynin or Tolterodine the amount is generally from about 2.5 to about 20 mg., and in an embodiment the amount is from about 5 to about 15 mg., whilst in an example the amount is 10 mg.

Hydroxypropylmethyl cellulose may be replaced with other sustained release vehicles. The amount and viscosity of each should be selected to provide a sustained release of the drug over a period of 24 hours.

The improvement of the invention in all aspects provides a post-menopausal woman with a protection against unwanted urination due to the frequent loss of muscle or sphincter control that accompanies the female aging process. A common example of this problem is leakage following a sneeze or a cough.

### EXAMPLE I

Using conventional tablet excipients and techniques, a rapidly dissolving tablet is provided which contains 3.9 mg. oxybutynin and 20 mg. fluoxetine. The tablet provides a mature, post-menopausal woman with enhanced relief against incontinence vis a vis a tablet without the fluoxetine.

### EXAMPLE II

Using conventional tablet excipients and techniques, a rapidly dissolving tablet is provided which contains 3.9 mg. oxybutynin and 20 mg. paroxetine. The tablet provides a mature, post-menopausal woman with enhanced relief against incontinence vis a vis a tablet without the paroxetine.

### EXAMPLES III-IV

Oral sustained release technology is exemplified by Guitard Example I which discloses “[a] therapeutic oxybutynin composition for administering to a patient *** prepared as follows: First, 103 grams of oxybutynin hydrochloride was dissolved in 1200 ml (milliliters) of anhydrous ethanol. Separately, 2,280 g of polyethylene oxide of 200,000 weight-average molecular weight, 150 g of hydroxypropylmethylcellulose of 9,200 average-number molecular weight and 450 g of sodium chloride were dry blended in a conventional blender for 10 minutes to yield a homogenous blend. Next, the oxybutynin ethanol solution was added slowly to the blend, with the blender continuously blending until all the ingredients were added to the three component dry blend, with the blending continued for another 8 to 10 minutes. The blended wet composition was passed through a 16 mesh screen and dried overnight at a room temperature of 72[deg] F. (22.2[deg]). Then, the dry granules were passed through a 20 mesh screen, 18 g of magnesium stearate was added, and all the ingredients blended again for 5 minutes. The fresh granules are ready for formulation into a therapeutic oxybutynin composition. The therapeutic composition comprises 3.4 wt % oxybutynin hydrochloride, 76 wt % polyethylene oxide of 200,000 weight-average molecular weight, 5 wt % of hydroxypropylmethylcellulose of 9,200 average-number molecular weight, 15 wt % sodium chloride, and 0.6 wt % magnesium stearate.” In accordance with the present invention, a sustained release tablet is provided by doubling the amounts of the two drug ingredients of Example I and II and incorporating this combination of drugs in place of the oxybutynin of Example I of Guittard. Each of the two tablets provides a 24 hour period of relief for incontinence.

### EXAMPLE V

Using conventional tablet excipients and techniques, a rapidly dissolving tablet is provided which contains 4.0 mg. Tolterodine and 20 mg. fluoxetine. The tablet provides a mature, post-menopausal woman with enhanced relief against incontinence vis a vis a tablet without the fluoxetine.

### EXAMPLE VI

Using conventional tablet excipients and techniques, a rapidly dissolving tablet is provided which contains 4.0 mg. Tolterodine and 20 mg. paroxetine. The tablet provides a mature, post-menopausal woman with enhanced relief against incontinence vis a vis a tablet without the paroxetine.

### EXAMPLES VII-VIII

Using the sustained release technology of Examples III-IV, a sustained release tablet is provided by doubling the amounts of the two drug ingredients of Example V and VI and otherwise following the procedure used in Examples III-IV. Each of the two tablets provides a 24 hour period of relief for incontinence.

### EXAMPLE IX

As a control, Example I of Quan is set forth: “Oxybutynin free base, pKa=10.3, is a strongly basic drug administered transdermally for antispasmodic and anticholinergic therapy. Matrix patches containing varying amounts of oxybutynin free base and penetration enhancers were prepared and tested as described above. The matrix systems consisted of 5 to 20% by weight of oxybutynin free base and 0 to 20% by weight of the enhancer contained in a medical grade acrylic copolymer adhesive.

“The matrix formulations were prepared as follows. First, the solids content of the adhesive was determined by weighing a small amount of the adhesive solution in a preweighed aluminum dish. The solvent was evaporated by overnight drying in a convection oven maintained at 80. degree. C. and the weight of the residue (dry adhesive) and percent solid adhesive content of the solution were determined. Once the solids content was determined, a known weight of the acrylic copolymer adhesive solution was weighed into a glass bottle. From the weight of the adhesive solution and the percent solid adhesive content, the amount of adhesive in the solution was calculated. Oxybutynin free base and enhancer were added to the bottle in proportions to yield the selected final composition. The bottle was then tightly capped, sealed with laboratory film, and rotated overnight until all ingredients had completely dissolved and the resultant solution was visually clear.

“Approximately 8 ml of the solution was then dispensed on a silanized polyester release liner and cast with a 10 mil gap casting knife. The casting was then dried in a convection oven at 70.degree. C. for 15 minutes to evaporate the solvent and to yield a dried film approximately 0.002 inch thick. A 0.003 inch thick polyethylene backing film was laminated onto the dried adhesive film with a rubber roller. These matrix laminates were then used to conduct in vitro skin flux studies that showed satisfactory results as explained in Table 1 of Quan.”.

The transdermal matrix for the delivery of oxybutynin of Example 1 of Quan is modified by incorporating therein 40 mg. of fluoxetine. Comparable results are achieved to those of Quan for patients other than post-menopausal women where the present invention provides a better retardation of active bladder response based upon weakened musculature.

### EXAMPLE X

The transdermal matrix for the delivery of oxybutynin of Example I of Quan is modified by incorporating therein 40 mg. of paroxetine. Comparable results are achieved to those of Quan for patients other than post-menopausal women where the present invention provides a better retardation of active bladder response based upon weakened musculature.

### EXAMPLES XI-XII

By replacing an equal amount of Tolterodine for the oxybutynin of Examples XIII and IX, a transdermal medication particularly suited for post-menopausal women is achieved that is designed to provide superior relief against active bladder caused by a weakened musculature.

### EXAMPLES XIII

The Waki et al. application discloses that “1.0 part of oxybutynin hydrochloride was dissolved in 200.0 parts of isoprapanol as the solvent, and then 20.0 parts of N-vinyl acetamide copolymer (PNVA GE167, a product of Showa Denko K.K.), 1.0 part of synthetic aluminum silicate and 1.0 part of borax were added and stir-mixed. The mixture solution containing 62.0 parts of glycerin and 15.0 parts of propylene glycol were added and continuously stirred.

“The solvent-type plaster with the desirable viscosity for the plaster is spread out over the non-woven fabric, then solvent is removed by heat drying (solvent drying) and the strippable film made of polyester was adhered. This was cut into the desirable size to obtain the transdermal absorption preparation containing oxybutynin hydrochloride.”

By replacing the oxybutynin of the quoted Waki example with a combination of each of the drugs as set forth in Examples XIII-XI, a superior overall medication is contemplated for post-menopausal women with a weakened musculature.

What is claimed is:

1. In the method of providing a post-menopausal female patient with a weakened musculature in the area of the urinary tract with an improved resistance to active bladder reaction via the oral delivery of oxybutynin, the improvement which comprises the coadministration therewith of an effective amount of FLUOXETINE whereby there is an enhanced resistance to said active bladder reaction.

2. The method of claim 1 wherein said coadministration is provided orally.

3. The method of claim 2 wherein the oral administration is via a sustained release vehicle to provide a 24 hour period of relief from active bladder reaction.

4. The method of claim 1 wherein said coadministration is from a transdermal patch.

5. In the method of providing a post-menopausal female patient with a weakened musculature in the area of the urinary tract with an improved resistance to active bladder reaction via the oral delivery of oxybutynin, the improvement which comprises the coadministration therewith of an effective amount of paroxetine whereby there is an enhanced resistance to said active bladder reaction.

6. The method of claim 6 wherein said coadministration is provided orally.

7. The method of claim 7 wherein the oral administration is via a sustained release vehicle to provide a 24 hour period of relief from active bladder reaction.

8. The method of claim 5 wherein said coadministration is from a transdermal patch.

9 In the method of providing a post-menopausal female patient with a weakened musculature in the area of the urinary tract with an improved resistance to active bladder reaction via the oral delivery of Tolterodine, the improvement which comprises the coadministration therewith of an effective amount of fluoxetine whereby there is an enhanced resistance to said active bladder reaction.

10. The method of claim 9 wherein said coadministration is provided orally.

11. The method of claim 10 wherein the oral administration is via a sustained release vehicle to provide a 24 hour period of relief from active bladder reaction.

12. The method of claim 11 wherein said coadministration is from a transdermal patch.

13. In the method of providing a post-menopausal female patient with a weakened musculature in the area of the urinary tract with an improved resistance to active bladder reaction via the oral delivery of Tolterodine, the improvement which comprises the coadministration therewith of an effective amount of paroxetine whereby there is an enhanced resistance to said active bladder reaction.

14. The method of claim 13 wherein said coadministration is provided orally.

15. The method of claim 14 wherein the oral administration is via a sustained release vehicle to provide a 24 hour period of relief from active bladder reaction.

16. The method of claim 13 wherein said coadministration is from a transdermal patch.

17. A composition suitable for providing a post-menopausal female patient with a weakened musculature in the area of the urinary tract with an improved resistance to active bladder reaction via the oral delivery of oxybutynin, the improvement which comprises the providing a dosage form for the administration of an effective amount of paroxetine whereby there is an enhanced resistance to said active bladder reaction.

18. The composition of claim 17 in oral form.

19. The composition of claim 18 in a sustained release vehicle to provide a 24 hour delivery to the patient.

20. A transdermal patch containing the medication of claim 17.

21. A composition suitable for providing a post-menopausal female patient with a weakened musculature in the area of the urinary tract with an improved resistance to active bladder reaction via the oral delivery of oxybutynin, the improvement which comprises the providing a dosage form for the administration of an effective amount of fluoxetine whereby there is an enhanced resistance to said active bladder reaction.

22. The composition of claim 20 in oral form.

23. The composition of claim 20 in a sustained release vehicle to provide a 24 hour delivery to the patient.

24. A transdermal medication of claim 20.

25. A composition suitable for providing a post-menopausal female patient with a weakened musculature in the area of the urinary tract with an improved resistance to active bladder reaction via the oral delivery of Tolterodine, the improvement which comprises the providing a dosage form for the administration of an effective amount of fluoxetine whereby there is an enhanced resistance to said active bladder reaction.

26. The composition of claim 25 in oral form.

27. The composition of claim 25 in a sustained release vehicle to provide a 24 hour delivery to the patient.

28. A transdermal patch containing the medication of claim 25.

29. A composition suitable for providing a post-menopausal female patient with a weakened musculature in the area of the urinary tract with an improved resistance to active bladder reaction via the oral delivery of Tolterodine, the improvement which comprises the providing a dosage form for the administration of an effective amount of paroxetine whereby there is an enhanced resistance to said active bladder reaction.

30. The composition of claim 29 in oral form.

31. The composition of claim 29 in a sustained release vehicle to provide a 24 hour delivery to the patient.

32. A transdermal patch containing the medication of claim 29.
